(19) United States
(12) Patent Application Publication
(10) Pub. No.: US 2023/0381356 A1
(43) Pub. Date: Nov. 30, 2023

(54) MICROBIOLOGICAL TRANSPORT MEDIUM AND METHODS OF USING THE SAME

(71) Applicant: Texas Health Resources, a Texas non-profit corporation, Arlington, TX (US)

(72) Inventor: Derek ORBACH, Fort Worth, TX (US)

(73) Assignee: Texas Health Resources, a Texas non-profit corporation, Arlington, TX (US)

(21) Appl. No.: 18/448,809

(22) Filed: Aug. 11, 2023

Related U.S. Application Data
(62) Division of application No. 17/213,030, filed on Mar. 25, 2021, now abandoned.

(51) Int. Cl. A61L 2/18; C12N 7/00; C12Q 1/6806

(52) U.S. Cl. CPC A61L 2/18; C12N 7/00; C12Q 1/6806; C12N 2770/18063; A61L 2101/38

(57) ABSTRACT

The present disclosure relates to the field of media for use in microbiological applications, and particularly in relation sample collection, transport, preparation, and storage. The disclosed media inactivate pathogenic (e.g., viral or bacterial) samples to allow for safe handling and storage, while simultaneously preserving nucleic acids for assessment.

### CROSS-REFERENCE TO RELATED APPLICATION

This application is a divisional application of U.S. application Ser. No. 17/213,030, filed Mar. 25, 2021, and which is entirely incorporated herein by reference.

### FIELD OF INVENTION

The present disclosure relates to the field of media for use in microbiological applications, and particularly in relation sample collection, preparation, transportation, and storage. The disclosed media inactivate pathogenic (e.g., viral, bacterial, prion) samples to allow for safe handling and storage, while simultaneously preserving nucleic acids for assessment.

### BACKGROUND

The following discussion is merely provided to aid the reader in understanding the disclosure and is not admitted to describe or constitute prior art thereto.

An outbreak of respiratory illness of unknown etiology in Wuhan City, Hubei Province, China was initially reported to the World Health Organization (WHO) on Dec. 31, 2019. Chinese authorities identified a novel coronavirus (SARS-CoV-2), which had resulted in thousands of confirmed human infections in multiple provinces throughout China. Within months, the virus had spread globally, causing severe infections and death and resulting in an unprecedented strain on health systems across the world.

Early in the global pandemic laboratory supplies such as PPE, transport media, disposables, various molecular assays, and culture media became increasingly difficult to secure. There was a growing interest in the medical community for large scale testing to be able to identify, track, and contact trace those that may have been infected with the virus. Although there was an early effort to create criteria for testing patients under investigation (PUIs) to stem hemorrhaging of already scarce supplies, increases in testing and sample collection were highly sought after and inevitably caused mass shortages. Supply of standard transport media (e.g., Universal Transport Media (UTM) and Viral Transport Media (VTM)), was quickly depleted, and concerns regarding how to safely handle patient samples rose to the forefront.

High throughput instruments were useful in meeting the need and demand for large-scale community testing; however, the instruments could only be used in a Biosafety Level 2 laboratory (BSL-2), without the aid of negative air pressure. Moreover, testing samples with viable SARS-CoV-2 in a BSL-2 required the use of N95 respirators, lab coats, wrap around impermeable gowns, and face shields; items that were in scarce supply. Concerns about laboratory acquired infections (LAIs) with SARS-CoV-2 mounted, and a need to inactivate the virus before handling became paramount.

Although several inactivation methods and guidance were provided by the molecular testing companies, those methods, which included heat (60° C. for 30 minutes), Guanidine Isothiocyanate (1.4M GITC in Tris buffer), and the combination of both 1.4M GITC TRIS and heat, were ultimately lacking. Indeed, it was determined that GITC alone at or near saturation (˜3.5M), did not effectively inactivate Pixuna virus (an enveloped Alphavirus surrogate; see Darnell, Evaluation of Inactivation Methods for Severe Acute Respiratory Syndrome Coronavirus in Noncellular Blood Products, Transfusion, 2006, 46(10):1770-1777), and a saturated GITC solution ˜3.5M alone does not inactivate Ebola Virus (Non-enveloped Virus; see Smither, Buffer AVL Alone Does Not Inactivate Ebola Virus in a Representative Clinical Sample Type, Journal of Clinical Microbiology, 2015, 53(10:3148-3154). As such, there were reasonable concerns that standard transportation media would not inactivate SARS-CoV-2 or allow for safe and efficient sample analysis.

Thus, there remains a need for a microbiology transport medium that can inactivate pathogens in samples obtained for patient testing.

### SUMMARY

Described herein are microbiology transport media that effectively and efficiently inactivate a broad spectrum of pathogens (e.g., bacterial or viral, and including SARS-CoV-2) and/or prions, which allows for safe handling and storage of samples, even in low biosafety levels with a range of open platform molecular instruments.

In one aspect, the present disclosure provides microbiology transport media, comprising: (a) a chaotropic agent; (b) a buffer; (c) a chelating agent; (d) a detergent; and (e) water.

The chaotropic agent can be selected from the group consisting of guanidine isothiocyanate, urea, lithium perchlorate, lithium acetate, phenol, thiourea, and guanidium chloride. In some embodiments, the chaotropic agent is guanidine isothiocyanate. The chaotropic agent can be present in an amount of about 3 M to about 5 M. In some embodiments, the chaotropic agent is present in an amount of about 4 M.

The buffer can be selected from the group consisting of Tris, sodium citrate/citrate buffer, L-glycine, acetate, borate, diethanolamine, carbonate (sodium), phosphate, MOPS (2-(N-morpholino)ethanesulfonic acid), bis-tris methane, ADA (N-(2-acetamido)iminodiacetic acid), bis-tris propane, PIPES (piperazine-N,N′-bis(2-ethanesulfonic acid)), ACES (N-(2-acetamido)-2-aminoethanesulfonic acid), MOPSO (3-morpholinopropanesulfonic acid), cholamine chloride, BES (N,N-Bis(2-hydroxyethyl)-2-aminoethanesulfonic acid), TES (2-[(2-hydroxy-1,1-bis(hydroxymethyl)ethyl)amino]ethanesulfonic acid), HEPES (4-(2-hydroxyethyl)-1-piperazineethanesulfonic acid), DIPSO (3-(N,N-Bis[2-hydroxyethyl]amino)-2-hydroxypropanesulfonic acid), MOBS (4-(N-morpholino)butanesulfonic acid), acetamindoglycine, TAPSO (2-hydroxy-3-[tris(hydroxymethyl)methylamino]-1-propanesulfonic acid), TEA (N,N-diethylethanamine), POPSO (piperazine-1,4-bis(2-hydroxypropanesulfonic acid) dihydrate), HEPPSO (4-(2-Hydroxyethyl)piperazine-1-ethanesulfonic acid), HEPPS (3-[4-(2-Hydroxyethyl)piperazin-1-yl]propane-1-sulfonic acid), tricine, glycinamide, glycylglycine, HEPBS (N-(2-Hydroxyethyl)piperazine-N′-(4-butanesulfonic acid)), bicine, TAPS ([tris(hydroxymethyl)methylamino]propanesulfonic acid), AMPSO (N-(1,1-Dimethyl-2-hydroxyethyl)-3-amino-2-hydroxypropanesulfonic acid), CAPS (N-cyclohexyl-3-aminopropanesulfonic acid), CABS (4-(cyclohexylamino)-1-butanesulfonic acid), and CHES (N-(cyclohexylamino)ethanesulfonic acid). In some embodiments, the buffer is Tris. The buffer can be present in an amount of about 0.3 M to about 0.5 M. In some embodiments, the buffer is present in an amount of about 0.4 M.

The chelating agent can be selected from the group consisting of ethylenediaminetetraacetic acid (EDTA); ethyleneglycol-bis(β-aminoethyl)-N,N,N′,N′-tetraacetic acid; ethylene glycol-bis(3-aminoethyl ether)-N,N,N′,N′-tetraacetic acid tetrasodium salt; 1,2-bis(o-aminophenoxy)ethane-N,N,N′,N′-tetraacetic acid; 1,2-bis(2-aminophenoxy)ethane-N,N,N′,N′-tetraacetic acid; and deferoxamine mesylate. In some embodiments, the chelating agent is EDTA. The chelating agent can be present in an amount of about 15 mM to about 35 mM. In some embodiments, the chelating agent is present in an amount of about 25 mM.

The detergent can be selected from the group consisting of Triton X-100, lithium dodecyl sulfate, sodium dodecyl sulfate, sodium lauryl sulfate, lithium lauryl sulfate, potassium lauryl sulfate, DDM (n-dodecyl beta-D-maltoside), digitonin, Tween 20, Tween 80, Chaps (3-[(3-Cholamidopropyl)dimethylammonio]-1-propanesulfonate), deoxycholate, cholate, and sarkosyl. In some embodiments, the detergent is Triton X-100. The detergent can be present in an amount of about 5% to about 15%. In some embodiments, the detergent is present in an amount of about 10%.

The water can be RNase free and/or DNase water.

The media can further comprise N-acetyl cysteine.

In some embodiments, the microbiology transport media, comprises: (a) 4 M guanidine isothiocyanate; (b) 0.4 M Tris hydrochloride (HCl) at about pH 8.0; (c) 25 mM ethylenediaminetetraacetic acid (EDTA) at about pH 8.0; (d) 10% Triton X-100; and (e) water. In such embodiments, the media may further comprise N-acetyl cysteine. In such embodiments, the water can be RNase free and/or DNase water.

In another aspect, the present disclosure also provides methods of handling a pathogen-containing sample, comprising obtaining a sample from a subject, wherein the sample contains or is believed to contain a pathogenic microorganism, and contacting the sample with any microbiology transport medium disclosed herein (e.g., a microbiology transport medium of the foregoing aspect or embodiments).

The sample can be obtained by a nasal swab, a nasopharyngeal swab, an oropharyngeal swab, or a bronchoalveolar lavage (BAL). The sample can be or comprise sputum, saliva, mucus, blood, plasma, serum, or tissue. Indeed, the sample may be any bodily fluid and the sample type is not particularly limited.

The pathogenic microorganism can be a virus (e.g., SARS-CoV-2), a bacterium, or a parasite. In some embodiments, the sample may additionally or alternatively contain or comprise a prion.

In some embodiments, the sample may be contacted with the microbiology transport medium after initially being placed, transported, or stored in a different medium, thereby inactivating, decontaminating, and/or sterilizing the sample. In some embodiments the sample may be initially contacted with the microbiology transport medium and not placed, transported, or stored in a different medium.

The disclosed methods may further comprise transporting, pipetting, and/or aerosolizing the sample after it has been contacted with the microbiology transport medium.

The disclosed methods may further comprise detecting or quantifying nucleic acids in the sample after it has been contacted with the microbiology transport medium.

In another aspect, the present disclosure also provides methods of sterilizing or decontaminating a surface, comprising contacting a surface with any microbiology transport medium disclosed herein (e.g., a microbiology transport medium of the first aspect or embodiments).

The surface may be infected with a bacterium, parasite, or a virus. Additionally or alternatively, the surface may be contaminated with a prion.

The present disclosure also provides microbiology transport media (e.g., a microbiology transport medium of the first aspect or embodiments) for use in handling, storing, inactivating, preserving, sterilizing or denaturing a biological sample.

The present disclosure also provides microbiology transport media (e.g., a microbiology transport medium of the first aspect or embodiments) for use in sterilizing or decontaminating a surface.

The foregoing general description and following detailed description are exemplary and explanatory and are intended to provide further explanation of the disclosure as claimed. Other objects, advantages, and novel features will be readily apparent to those skilled in the art from the following detailed description of the disclosure.

### DETAILED DESCRIPTION

The present disclosure provides a novel microbiology transport medium and methods of using the same to inactivate, store, transport, and safely handle biological samples that contain (or are suspected of containing) pathogenic microorganisms (e.g., virus, parasite, or bacterium) and/or prions. The disclosed media can also be used as a disinfectant or decontaminant of various surfaces (e.g., molecular testing equipment, surgical equipment/instruments/trays, laboratory bench space, etc.), as the disclosed media can inactivate, denature, lyse, and/or kill a broad spectrum of microorganisms and even denature and inactive prions.

### I. Definitions

It is to be understood that the terminology used herein is for describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the term “comprising” is intended to mean that the compositions and methods include the recited elements, but does not exclude others.

For the purposes of the present disclosure, the terms “media” and “mediums” may be used interchangeably as the plural forms of the term “medium.” Similarly, the disclosed microbiology transport media/medium may be interchangeably referred to simply as “the disclosed media” or “the disclosed medium.”

### II. Microbiology Transport Medium

The present disclosure provides novel microbiology transport media. In general, the disclosed media comprise: (a) a chaotropic agent; (b) a buffer; (c) a chelating agent; (d) a detergent; and (e) water. Optionally, the disclosed media can also comprise N-acetyl cysteine. The water may be RNase and/or DNase free water. The amounts of each component and the identity of the component within each category (i.e., chaotropic agent, buffer, chelating agent, detergent) may vary, as discussed in more detail below.

For example, in one embodiment, the microbiology transport medium can comprise: (a) about 4 M guanidine isothiocyanate (GITC); (b) about 0.4 M Tris hydrochloride (HCl) at about pH 8.0; (c) about 25 mM ethylenediaminetetraacetic acid (EDTA) at about pH 8.0; (d) about 10% Triton X-100; and (e) water. Those of skill in the art will understand, however, that this is merely one exemplary embodiment and the scope of the disclosure is not so limited.

### A. Chaotropic Agents

A chaotropic agent is, in general, understood to be a compound that can disrupt the hydrogen bonding network between water molecules. This has an effect in the stability of the native state of other molecules in the solution, mainly macromolecules, such as proteins and nucleic acids, by weakening the hydrophobic effect. Indeed, chaotropic solutes increase the entropy of a system by interfering with intermolecular interactions mediated by non-covalent forces such as hydrogen bonds, van der Waals forces, and hydrophobic effects. Numerous chaotropic agents are known in the art. The disclosed microbiology transport media contain at least one chaotropic agent.

For the purposes of the present disclosure, the chaotropic agent can be, but is not limited to guanidine isothiocyanate (GITC), urea, lithium perchlorate, lithium acetate, phenol, thiourea, and guanidium chloride. Guanidine isothiocyanate is a preferred chaotropic agent.

Guanidine isothiocyanate (GITC or guanidinium thiocyanate or GTC) is a chemical compound that can serve as a protein denaturant and a nucleic acid (e.g., DNA or RNA) protector, as well as being a chaotropic agent. GITC has been assigned the CAS number 593-84-0, a chemical formula of C₂H₆N₄S, and the following chemical structure:

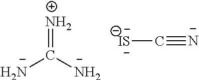

The chaotropic agent (e.g., GITC) in the disclosed media can is present in an amount of about 1 M to about 10 M, about 2 M to about 8 M, or about 3 M to about 5 M, and any value in between. In other words, the amount/concentration of the chaotropic agent (e.g., GITC) can be 1.00 M, 1.25 M, 1.5 M, 1.75 M, 2.00 M, 2.25 M, 2.5 M, 2.75 M, 3.00 M, 3.25 M, 3.5 M, 3.75 M, 4.00 M, 4.25 M, 4.5 M, 4.75 M, 5.00 M, 5.25 M, 5.5 M, 5.75 M, 6.00 M, 6.25 M, 6.5 M, 6.75 M, 7.00 M, 7.25 M, 7.5 M, 7.75 M, 8.00 M, 8.25 M, 8.5 M, 8.75 M, 9.00 M, 9.25 M, 9.5 M, 9.75 M, or 10 M or any value in between. In some embodiments, the chaotropic agent (e.g., GITC) is present in an amount of about 4 M or exactly 4 M.

### B. Buffer

Buffers are, in general (i) weak acids and their conjugate base or (ii) weak bases and their conjugate acid, which help maintain the pH of an aqueous solution. If an acid or a base is added to a buffered solution, the pH of the buffered solution will not change significantly. Numerous commonly used buffers are known in the art. The disclosed microbiology transport media contain at least one buffer.

For the purposes of the present disclosure, the buffer can be, but is not limited to Tris, sodium citrate/citrate buffer, L-glycine, acetate, borate, diethanolamine, carbonate (sodium), phosphate, MOPS (2-(N-morpholino)ethanesulfonic acid), bis-tris methane, ADA (N-(2-acetamido)iminodiacetic acid), bis-tris propane, PIPES (piperazine-N,N′-bis(2-ethanesulfonic acid)), ACES (N-(2-acetamido)-2-aminoethanesulfonic acid), MOPSO (3-morpholinopropanesulfonic acid), cholamine chloride, BES (N,N-bis(2-hydroxyethyl)-2-aminoethanesulfonic acid), TES (2-[(2-hydroxy-1,1-bis(hydroxymethyl)ethyl)amino]ethanesulfonic acid), HEPES (4-(2-hydroxyethyl)-1-piperazineethanesulfonic acid), DIPSO (3-(N,N-Bis[2-hydroxyethyl]amino)-2-hydroxypropanesulfonic acid), MOBS (4-(N-morpholino)butanesulfonic acid), acetamindoglycine, TAPSO (2-hydroxy-3-[tris(hydroxymethyl)methylamino]-1-propanesulfonic acid), TEA (N,N-diethylethanamine), POPSO (piperazine-1,4-bis(2-hydroxypropanesulfonic acid) dihydrate), HEPPSO (4-(2-Hydroxyethyl)piperazine-1-ethanesulfonic acid), HEPPS (3-[4-(2-Hydroxyethyl)piperazin-1-yl]propane-1-sulfonic acid), tricine, glycinamide, glycylglycine, HEPBS (N-(2-Hydroxyethyl)piperazine-N′-(4-butanesulfonic acid)), bicine, TAPS ([tris(hydroxymethyl)methylamino]propanesulfonic acid), AMPSO (N-(1,1-Dimethyl-2-hydroxyethyl)-3-amino-2-hydroxypropanesulfonic acid), CAPS (N-cyclohexyl-3-aminopropanesulfonic acid), CABS (4-(cyclohexylamino)-1-butanesulfonic acid), and CHES (N-(cyclohexylamino)ethanesulfonic acid). Tris and Tris HCl are preferred buffers.

Tris (i.e., tris(hydroxymethyl)aminomethane, tromethamine, or THAM) is an organic compound that is extensively used in biochemistry and molecular biology as a component of buffer solutions. It contains a primary amine that undergoes the reactions associated with typical amines, e.g. condensations with aldehydes. Tris and salts thereof (e.g., Tris HCl) are commonly used as buffering agents, but may also have inhibitory effects on some enzymes and possess some chelating activity. Tris has been assigned the CAS number 77-86-1 (free base) or 1185-53-1 (hydrochloride), a chemical formula of (HOCH₂)₃CNH₂, and the following chemical structure:

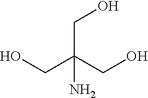

The buffer (e.g., Tris) in the disclosed media can is present in an amount of about 0.1 M to about 1.0 M, about 0.2 M to about 0.8 M, or about 0.3 M to about 0.5 M, and any value in between. In other words, the amount/concentration of the buffer (e.g., Tris) can be 0.1 M, 0.125 M, 0.15 M, 0.175 M, 0.2 M, 0.225 M, 0.25 M, 0.275 M, 0.3 M, 0.325 M, 0.35 M, 0.375 M, 0.4 M, 0.425 M, 0.45 M, 0.475 M, 0.5 M, 0.525 M, 0.55 M, 0.575 M, 0.6 M, 0.625 M, 0.65 M, 0.675 M, 0.7 M, 0.725 M, 0.75 M, 0.775 M, 0.8 M, 0.825 M, 0.85 M, 0.875 M, 0.9 M, 0.925 M, 0.95 M, 0.975 M, or 0.1.0 M or any value in between. In some embodiments, the buffer (e.g., Tris) is present in an amount of about 0.4 M or exactly 0.4 M.

### C. Chelating Agent

A chelating agent (i.e., chelants, chelators, chelating agents, or sequestering agents) is a chemical compound that reacts with metal ions to form a stable, water-soluble complex. Chelating agents often have a ring-like center, which can forms at least two bonds with a metal ion. Numerous commonly used chelating agents are known in the art. The disclosed microbiology transport media contain at least one chelating agent.

For the purposes of the present disclosure, the chelating agent can be, but is not limited to ethylenediaminetetraacetic acid (EDTA); ethyleneglycol-bis(β-aminoethyl)-N,N,N′,N′-tetraacetic acid; ethylene glycol-bis(R-aminoethyl ether)-N,N,N′,N′-tetraacetic acid tetrasodium salt; 1,2-bis(o-aminophenoxy)ethane-N,N,N′,N′-tetraacetic acid; 1,2-bis(2-aminophenoxy)ethane-N,N,N′,N′-tetraacetic acid; and deferoxamine mesylate. EDTA is a preferred chelating agent.

Ethylenediaminetetraacetic acid (EDTA) is an aminopolycarboxylic acid that is generally in the form of a white, water-soluble solid is widely used to bind to iron and calcium ions. EDTA binds these ions as a hexadentate chelating agent. EDTA is produced as several salts, notably disodium EDTA, sodium calcium edetate, and tetrasodium EDTA. EDTA has been assigned the CAS number 60-00-4 (free acid) or 6381-92-6 (dihydrate disodium salt), a chemical formula of [CH₂N(CH₂CO₂H)₂]₂, and the following chemical structure:

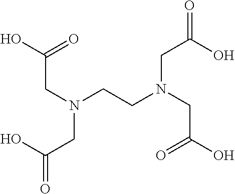 [SMILES: O=C(O)CN(CCN(CC(=O)O)CC(=O)O)CC(=O)O]

The chelating agent (e.g., EDTA) in the disclosed media can is present in an amount of about 5 mM to about 55 mM, about 10 mM to about 45 mM, or about 15 mM to about 35 mM, and any value in between. In other words, the amount/concentration of the chelating agent (e.g., EDTA) can be 5.0 mM, 5.5 mM, 6.0 mM, 6.5 mM, 7.0 mM, 7.5 mM, 8.0 mM, 8.5 mM, 9.0 mM, 9.5 mM, 10.0 mM, 10.5 mM, 11.0 mM, 11.5 mM, 12.0 mM, 12.5 mM, 13.0 mM, 13.5 mM, 14.0 mM, 14.5 mM, 15.0 mM, 15.5 mM, 16.0 mM, 16.5 mM, 17.0 mM, 17.5 mM, 18.0 mM, 18.5 mM, 19.0 mM, 19.5 mM, 20.0 mM, 20.5 mM, 21.0 mM, 21.5 mM, 22.0 mM, 22.5 mM, 23.0 mM, 23.5 mM, 24.0 mM, 24.5 mM, 25.0 mM, 25.5 mM, 26.0 mM, 26.5 mM, 27.0 mM, 27.5 mM, 28.0 mM, 28.5 mM, 29.0 mM, 29.5 mM, 30.0 mM, 30.5 mM, 31.0 mM, 31.5 mM, 32.0 mM, 32.5 mM, 33.0 mM, 33.5 mM, 34.0 mM, 34.5 mM, 35.0 mM, 35.5 mM, 40.0 mM, 45.0 mM, or 50.0 mM, or any value in between. In some embodiments, the chelating agent (e.g., EDTA) is present in an amount of about 25 mM or exactly 25 mM.

### D. Detergent

Detergents are, in general, surfactants or mixtures of surfactants, which generally are amphiphilic: partly hydrophilic (polar) and partly hydrophobic (non-polar). Their dual nature facilitates the mixture of hydrophobic compounds (like oil and grease) with water. Numerous commonly used detergents are known in the art. The disclosed microbiology transport media contain at least one detergent.

For the purposes of the present disclosure, the detergent can be, but is not limited to Triton X-100, lithium dodecyl sulfate, sodium dodecyl sulfate, sodium lauryl sulfate, lithium lauryl sulfate, potassium lauryl sulfate, DDM (n-dodecyl beta-D-maltoside), digitonin, Tween 20, Tween 80, Chaps (3-[(3-Cholamidopropyl)dimethylammonio]-1-propanesulfonate), deoxycholate, cholate, and sarkosyl. Triton X-100 is a preferred detergent.

Triton X-100 is a nonionic surfactant that has a hydrophilic polyethylene oxide chain (on average it has 9.5 ethylene oxide units) and an aromatic hydrocarbon lipophilic or hydrophobic group. Triton X-100 is a clear viscous fluid (less viscous than undiluted glycerol), and it is distantly related to Pluronic range of detergents marketed by BASF. The pluronics are triblock copolymers of ethylene oxide and propylene oxide with the ethylene oxide segments being more hydrophilic than the propylene oxide. Triton X-100 has been assigned the CAS number 9002-93-1, a chemical formula of C₁₄H₂₂O(C₂H₄O)ₙ, and the following chemical structure:

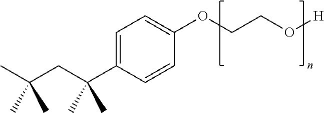

Triton X-100 has been shown to inactivate Pseudorabies Virus (an enveloped DNA virus), Bovine Viral Diarrheal Virus (Enveloped RNA virus), HIV (Enveloped RNA virus), and Xenotropic murine leukemia virus (Enveloped RNA virus). See Farcet et al., Development of a Triton X-100 Replacement for Effective Virus Inactivation in Biotechnology Processes, Engineering Reports, 2019, 1(5)). Indeed, Triton X-100 has been shown to achieve a 4-log reduction in <1 minute, at a final concentration of 1% (v/v). Triton X-100 quickly dissolves membranes, while leaving surface proteins intact.

The detergent (e.g., Triton X-100) in the disclosed media can is present in an amount of about 1% to about 25%, about 3% to about 20%, or about 5% to about 15%, and any value in between. In other words, the amount/concentration of the detergent (e.g., Triton X-100) can be 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, or 25%, or any value in between. In some embodiments, the detergent (e.g., Triton X-100) is present in an amount of about 10% or exactly 10%.

### E. Exemplary Embodiments of the Disclosed Media

The disclosed media can comprise: (a) a chaotropic agent in an amount between about 1 M to about 10 M; (b) a buffer in an amount between about 0.1 M to about 1.0 M; (c) a chelating agent in an amount between about 5 mM to about 55 mM; (d) a detergent in an amount between about 1% to about 25%; and (e) water.

In some embodiments, the disclosed media can comprise: (a) a chaotropic agent in an amount between about 3 M to about 5 M; (b) a buffer in an amount between about 0.3 M to about 0.5 M; (c) a chelating agent in an amount between about 15 mM to about 35 mM; (d) a detergent in an amount between about 5% to about 15%; and (e) water.

In some embodiments, the disclosed media can comprise: (a) a chaotropic agent in an amount of about 4 M; (b) a buffer in an amount of about 0.4 M; (c) a chelating agent in an amount of about 25 mM; (d) a detergent in an amount of about 10%; and (e) water.

In some embodiments, the chaotropic agent is GITC. In some embodiments, the buffer is Tris or Tris HCl. In some embodiments, the chelating agent is EDTA. In some embodiments, the detergent is Triton X-100. Due to the protein disrupting effect of GITC and the membrane disrupting effect of Triton X-100, formulations containing GITC and Triton have been shown to quickly inactivate SARS-CoV-2 in vitro (Scallan et al., Validation of a Lysis Buffer Containing 4 M Guanidinium Thiocyanate (GITC) Triton X-100 for Extraction of SARS-CoV-2 RNA for COVID-19 Testing: Comparison of Formulated Lysis Buffers Containing 4 to 6 M GITC, Roche External Lysis Buffer and Qiagen RTL Lysis Buffer, bioRxIv, 2020). The present disclosure shows that neither ingredient will disrupt downstream molecular testing on instruments that contain wash steps or with molecular testing in which extraction takes place first, either manually or in an automated fashion.

Optionally, the disclosed media can also comprise N-acetyl cysteine. Optionally, the water may be RNase and/or DNase free water.

Any of the transport media disclosed herein can be used for safely and effectively inactivating, denaturing, handling, or storing a biological sample (e.g., sputum, mucus, blood, serum, plasma, tissue, etc.) that contains or is believed to contain a pathogenic microorganism (e.g., virus, parasite, or bacterium) and/or a prion. Any of the transport media disclosed herein can also be used for sanitizing or decontaminating a surface, such as molecular testing equipment, surgical equipment/instruments/trays, laboratory bench space, etc.

### III. Making the Disclosed Medium

While the disclosed media may be prepared using any known or suitable method or procedure, they also can be prepared using the methodologies disclosed herein. For example, an exemplary embodiment of the disclosed microbiology transport medium, which comprises 4 M GITC, 0.4 M Tris HCl (pH 8.0), 25 mM EDTA (pH 8.), 10% Triton X-100, and RNase free water, can be prepared as follows:
1. Measure 472.64 grams Guanidine Isothiocyanate (GITC);
2. Mix with 400 ml 1M Tris pH 8.0 in a sterile flask;
3. Heat mixture in a 65° C. water bath until GITC is fully dissolved;
4. Add 50 ml 1M EDTA pH 8.0;
5. Add 100 ml Triton X-100;
6. Raise volume to 1 L with RNase free H₂O; and
7. Mix thoroughly.

Those skilled in the art will understand that the concentrations of the components and even the components themselves may be altered or adjusted as needed within the disclosed ranges and categories of compounds (e.g., chaotropic agents, buffers, chelating agents, and detergents), respectively.

### IV. Methods of Use

The present disclosure provides novel microbiology transport media that comprise a saturated solution of a chaotropic agent (e.g., GITC) and a detergent (e.g., Triton X-100) to overcome the supply challenges and to address safety concerns surrounding the storage, manipulation, and transport of patient samples potentially containing high concentrations of SARS-CoV-2. The formula was created to quickly inactivate SARS-CoV-2, and subsequently stabilize the released nucleic acids. Nucleases are quickly denatured by GITC, while the nucleic acids are protected from free hydronium ions utilizing Tris buffer. Divalent cations are sequestered by the addition of a chelating agent (e.g., EDTA), adding further protection from nuclease activity. Altogether, the disclosed media can render safe and stabilize samples containing a broad spectrum of pathogenic microorganisms (e.g., viruses, parasites, and bacteria) beyond SARS-CoV-2.

The disclosed media can destroy all microorganisms (bacteria, viruses, parasites, etc.) and even inactivate/decontaminate prions (i.e., proteinase resistant proteins, or PrPs) in a given sample, thereby completely sterilizing the sample. Additionally, the disclosed media can simultaneously inactivate all nucleases (RNase and DNase, etc.), which immediately stabilizes all nucleic acids (DNA and RNA) in the sample for downstream nucleic acid amplification testing. Further, all divalent cations (magnesium, calcium, zinc, etc.) which are crucial to nuclease function, are bound and chelated, rendering them unusable to nucleases. This multifaceted functionality allows the disclosed media to safely stabilize, protect, and preserve biological samples for further diagnostic and/or molecular assessment, while also protecting laboratory personnel from dangerous pathogens.

The media allows handling, pipetting, and aerosol generating procedures to be performed safely in a laboratory setting without the use of additional personal protective equipment (PPE) and while being in a BSL1 or BSL2 (or higher) facility. The disclosed media also allows for the safe transport of all specimens regardless of what pathogen may be contained therein.

The disclosed media are able to liquefy tissues and break down mucin, which allows for pipetting in automated instruments and analysis on automated instruments without the need for sample cleanup or processing. Indeed, a sample can be taken directly from a patient and placed into a container with the disclosed media, and then the media can be assessed on automated instruments for the presence of nucleic acids of a pathogenic microorganism of interest (e.g., bacteria or viruses, such as SARS-CoV-2) without cleanup or processing of the media/sample. This allows for multiple specimen types (such as sputum, saliva, mucus, blood, plasma, serum, or tissue), and multiple sampling methods (such as nasal swab, nasopharyngeal swab, oropharyngeal swab, or bronchoalveolar lavage) to be utilized in a more streamlined and/or automated fashion for analysis.

The media also allows other media types to be mixed into it, thus allowing it to act as a decontamination agent. For example, if a specimen is collected in universal transport media (UTM), it can then be transferred into the disclosed media to inactivate viruses, bacteria, or other pathogenic microorganisms. As such, highly dangerous samples that are collected in alternative media can be inactivated quickly without multiple heating steps or other procedures that may be more laborious, time-consuming, less efficient, and/or require specialized equipment or safety precautions (e.g., a BSL3 or BSL4 lab space). Additionally, added decontamination steps such as heating may degrade/damage nucleic acid, thus decreasing the sensitivity or downstream molecular testing, but the disclosed uses and methods do not raise the same concerns.

As shown in the Examples provided herein, the disclosed media has been shown to function adequately with the Abbott m2000, Abbott Alinity m, Biofire Film Array, Biofire Torch, and Cepheid GeneXpert. Thus, it is expected that the disclosed media will function adequately in any other similar instruments and assays, and the disclosed media may be used on all molecular equipment that includes a wash step, including the Roche Cobas, as well or in concert with extractors, including but not limited to the Biomerieux EMag, Biomerieux EZMag, Promega Maxwell, and others.

The media may also inactivate prions (PrPs), thus proving to be a promising wash for surgical equipment/instruments/trays prior to autoclaving and a broad spectrum decontaminant for other surfaces. This inactivation could be shown, for example, by adding the media in vitro to prion-infected homogenized brain samples, which are subsequently used to infect Monotypic neuronal GT1 cells. The Monotypic neuronal GT1 cells are then examined for evidence of PrPs. Absence of the evidence of PrPs would show denaturation of the PrP in the homogenized brain sample. Alternatively, recombinant PrP could be generated using E. coli. The PrP samples are then washed with OTE, and again used to infect Monotypic neuronal GT1 cells. Again, the absence of PrP would show that the PrPs are denatured/inactivated through contact with the disclosed media.

In view of all of the foregoing, the present disclosure provides methods of handling a pathogen-containing sample, comprising obtaining a sample from a subject, wherein the sample contains or is believed to contain a pathogenic microorganism, and contacting the sample with the microbiology transport medium disclosed herein.

The sample can be obtained by a nasal swab, a nasopharyngeal swab, an oropharyngeal swab, or a bronchoalveolar lavage (BAL). The sample can be or can comprise sputum, saliva mucus, blood, plasma, serum, or tissue.

The pathogenic microorganism in the sample may be a virus (e.g., SARS-CoV-2), a parasite, or a bacterium. Exemplary pathogenic microorganisms include, but are not limited to, SARS-CoV-2, SARS-CoV-1, MERS, common Coronaviruses, Influenza viruses (all subtypes), Respiratory Syncytial Virus, Rhinovirus, Bacillus spp. (including anthracis), Clostridium spp. (including perfringens, and botulinum), Clostridioides difficile, Pseudomonas spp., Burkholderia spp., Morganella spp., Providencia spp., Proteus spp., Yersinia spp. (including pestis), Fracisella spp. (including tularensis), all causative agents of Viral hemorrhagic fevers (including Filoviruses, and Arenaviruses), Variola major, Brucella spp., Salmonella spp., Escherichia spp. (including coli 0157:57, and all shiga toxin producing E. coli), Shigella spp., Vibrio spp., Coxiella spp., Chlamydia spp, Neisseria spp., Rickettsia spp., Alphaviruses, Cryptosporidium parvum, Trichomonas vaginalis, Trypanosoma brucei gambiense, Trypanosoma brucei rhodesiense, Babesia spp., Trypanosoma spp., Leishmania spp., and Plasmodium spp. While not technically a pathogenic microorganism, in some embodiments, the sample may contain a prion (i.e., PrP) including but not limited to, a prion causing CID and variants, a prion causing Gerstmann-Straussler-Scheinker disease, a prion causing Kuru, and a prion causing Fatal insomnia. These lists of pathogenic microorganisms and prions are understood to be non-limiting and ds not mention all the possible microorganisms and prions that are destroyed by the disclosed media. The media has been found to provide broad-spectrum sterilization of samples, instruments, and surfaces.

In some embodiments, the sample may be contacted with the disclosed microbiology transport medium after initially being placed, transported, or stored in a different medium, thereby inactivating, decontaminating, and/or sterilizing the sample upon contact with the disclosed microbiology medium. In other embodiments, the sample can be initially contacted with or placed into the disclosed microbiology transport medium and not placed, transported, or stored in a different medium.

In some embodiments, the disclosed methods of handling and/or storing a sample may further comprise transporting, pipetting, and/or aerosolizing the sample after it has been contacted with the disclosed microbiology transport medium. In some embodiment, the methods may further comprise detecting or quantifying nucleic acids in the sample after it has been contacted with the disclosed microbiology transport medium. For example, the methods may further comprise assessing a sample that has been stored in or contact with the disclosed media for the presence of a pathogenic microorganism. Such assessing may comprise molecular analysis and/or automated analysis to detect and/or quantify nucleic acids from a given pathogenic microorganism.

The present disclosure also provides methods of sterilizing or decontaminating a surface, comprising contacting a surface with the microbiology transport media disclosed herein. In some embodiments, the surface may be infected with a bacterium, a virus, or a parasite. In some embodiment, the surface may be contaminated with a prion.

The following examples are given to illustrate the present disclosure. It should be understood that the invention is not to be limited to the specific conditions or details described in these examples.

### EXAMPLES

### Example 1—Validation and Demonstration for SARS-CoV-2 Collection

This example describes the validation and demonstration of acceptable performance of the disclosed media for the collection of SARS-CoV-2 specimens. While the below validation study is for the Abbott m2000 SARS CoV-2 assay, the disclosed media may be used to transport all respiratory viral/pathogen specimens, and validation is exemplary of the use of the disclosed media on all molecular equipment before patient testing commences. Quality control of the disclosed media was performed on new Lots and shipments. Quality control contained respiratory viral RNA of the assay positive target, human RNase P DNA as the negative control, and a blank sample containing no nucleic acids. The disclosed media was used only after acceptable QC is recorded.

Materials

GITC/Triton X-100 Media (hereafter in this example, “the disclosed media”):
4M Guanidinium Thiocyanate
0.4M Tris HCl pH 8.0
25 mM EDTA pH 8.0
10% Triton X-100

### Experimental Design

Shielded RNA Standard Curve and Limit of Detection (LOD)

Serial dilutions were created using SeraCare AccuPlex SARS-CoV-2 shielded RNA positive controls. The initial concentration of viral RNA was 5000 copies/mL. This was diluted in the media, to provide viral concentrations of 250 copies/mL, 100 copies/mL, 25 copies/mL, and 10 copies/mL. Each dilution was tested on the Abbott m2000 five times and CN numbers were recorded. A standard curve was generated using average CN values versus viral copy concentration.

Correlation Study

Nasopharyngeal (NP) samples that were collected were initially tested using Abbott IDNow SARS-CoV-2 Assay, and Cepheid GeneXpert Infinity SARS-CoV-2 Assay, and subsequently tested for correlation on the Abbott m2000 SARS-CoV-2 assay utilizing the disclosed media.

The patients' initial samples were collected upon admission in Universal Transport Media™ (UTM®) from Copan Diagnostics and Remel and tested on either the Abbott IDNOW or the Cepheid GeneXpert Infinity. Later, both NP and Nasal swabs were again collected and immediately placed in disclosed media and transported to the microbiology laboratory. The samples were then loaded on the m2000. CN values and results were recorded.

Spiked Samples and Precision

Serial dilutions were created using SeraCare AccuPlex SARS-CoV-2 shielded RNA positive controls.

To provide middle and low concentration samples, the initial concentration of 5000 copies/ml was diluted to 500 copies/ml, 300 copies/ml, 100 copies/ml, and 50 copies/ml. Five spiked samples (20 samples total) were created at each concentration and tested on the Abbott m2000 instrument. These samples were stored at 2-8° C. and tested at 24-hour intervals for 72 hours (initial test, with two additional replicate studies).

Contrived negative samples were also created using SeraCare AccuPlex SARS-CoV-2 shielded RNA negative controls. The negative control was diluted in disclosed media and tested. The negative samples were also stored at 2-8° C. and tested at 24-hour intervals for 72 hours (initial test, with two additional replicate studies).

Results

Shielded RNA Standard Curve and Limit of Detection (LOD)

All serial dilution samples were detected, except for two of five samples at 10 copies/ml. Accordingly, 10 copies/ml was the empiric limit of detection for this assay utilizing the disclosed media.

Correlation Study

There were 26 samples of known SARS-CoV-2 positive patients tested. A combination of bilateral nasal and NP collection were studied. Of those, one bilateral nasal collection was found to be negative, and the other 25 samples were found to be positive (CN range 11.01 to 29.21) in agreement with other SARS-CoV-2 testing methodologies.

Nasal collection contained notably less viral RNA than the corresponding NP collection. CN values for bilateral nasal collections were much higher than those of the NP swab on the same patient. The only nasal collection that was negative could have been due to low viral count with this collection method, as the corresponding NP collection seemed to have a high CN value (26.97) and caused the nasal collection to fall below the limit of detection for this assay.

Precision Study

In the precision study utilizing the disclosed media, 90 replicate tests were performed with only one (1%) out of agreement with the expected results. 89 of 90 replicate tests (99%) agreed with the original expected result. The single outlier sample was tested 10 times, all of which retested positive, showing that the single test out of agreement may have been due to instrument failure, or experimental failure.

During the precision study, samples were stored at 2-8° C. for 72 hours and showed similar CN values with each test, showing that samples are stable for at least 72 hours after collection at 2-8° C.

Sensitivity: Sensitivity of the Abbott m2000 SARS-CoV-2 Assay was calculated to be 98%.

Specificity: Specificity of the Abbott m2000 SARS-CoV-2 Assay was calculated to be 100%.

Positive Predictive Value: Positive Predictive Value of the Abbott m2000 SARS-CoV-2 Assay was calculated to be 100%.

Negative Predictive Value: Negative Predictive Value of the Abbott m2000 SARS-CoV-2 Assay was calculated to be 96%.

Reportable Range:
SARS-CoV-2 Negative
SARS-CoV-2 Positive

### Summary

Of the 158 total specimens tested, only 2 samples did not agree with the expected results. The one sample in disagreement may have been due to experimental or instrument error, while the other showed nasal collection to be inferior to nasopharyngeal collection.

Specimens were shown to be adequate and acceptable for testing for up to 72 hours if stored at 2-8° C., and has a limit of detection of 10 copies/ml. In all studies, no carryover was observed.

### Conclusion

The disclosed media is acceptable for use in testing patients for SARS-CoV-2.

### Example 2—Validation and Demonstration of Media Performance on Abbott Alinity Platform

This example describes the validation and demonstration of acceptable performance of the disclosed media for the Abbott Alinity m SARS-CoV-2 PCR assay while utilizing the disclosed media as the terminal inactivating step. The Abbott Alinity m SARS-CoV-2 assay is a real-time reverse transcription polymerase chain reaction (rRT-PCR) test on the Abbott Alinity m System. The SARS-CoV-2 primer and probe sets are designed to detect RNA from SARS-CoV-2 in nasal, nasopharyngeal and oropharyngeal swabs or BAL from patients with signs and symptoms of infection who are suspected of COVID-19 by their health care provider.

Assay Method

The Alinity m SARS-CoV-2 assay is a real-time reverse transcriptase (RT) polymerase chain reaction (PCR) test intended for the qualitative detection of nucleic acid from the SARS-CoV-2 in nasal swabs, self-collected at a health care location or collected by a healthcare worker, nasopharyngeal (NP) and oropharyngeal (OP) swabs collected by a healthcare worker or bronchoalveolar lavage fluid (BAL) from patients suspected of COVID-19 by their health care provider. Testing is limited to laboratories certified under the Clinical Laboratory Improvement Amendments of 1988 (CLIA), 42 U.S.C. § 263a, to perform moderate or high complexity tests.

The assay is used for the identification of SARS-CoV-2 RNA. The SARS-CoV-2 RNA is generally detectable in respiratory specimens during the acute phase of infection. Positive results are indicative of the presence of SARS-CoV-2 RNA; clinical correlation with patient history and other diagnostic information is necessary to determine patient infection status. Positive results do not rule out bacterial infection or co-infection with other viruses. Laboratories within the United States and its territories are required to report all positive results to the appropriate public health authorities.

Negative results do not preclude SARS-CoV-2 infection and should not be used as the sole basis for patient management decisions. Negative results must be combined with clinical observations, patient history, and epidemiological information.

The Alinity m SARS-CoV-2 assay is intended for use by qualified and trained clinical laboratory personnel specifically instructed and trained in the techniques of real-time PCR and in vitro diagnostic procedures. The Alinity m SARS-CoV-2 assay is only for use under the Food and Drug Administration's Emergency Use Authorization.

The Abbott Alinity m SARS-CoV-2 assay is a dual target assay for the RdRp and N genes. An RNA sequence that is unrelated to the SARS-CoV-2 target sequence is introduced into each specimen at the beginning of sample preparation. This unrelated RNA sequence is simultaneously amplified by RT-PCR and serves as an internal control (IC) to demonstrate that the process has proceeded correctly for each sample.

The Abbott Alinity m SARS-CoV-2 assay detects the SARS-CoV-2 virus and IC target sequences through the use of target-specific fluorescent-labeled oligonucleotide probes. The probes do not generate a signal unless they are specifically bound to the amplified product. The two SARS-CoV-2-specific probes are labeled with the same fluorophore and the IC-specific probe is labeled with a different fluorophore, thus allowing for simultaneous detection of both SARS-CoV-2 and IC amplified products in the same reaction well.

The Abbott Alinity m SARS-CoV-2 assay is performed on the Abbott Alinity m System, which performs sample preparation, RT-PCR assembly, amplification, detection, and result calculation and reporting. All steps of the Alinity m SARS-CoV-2 assay procedure are executed automatically by the Alinity m System. Application parameters specific to the Abbott Alinity m SARS-CoV-2 assay are contained on an assay-specific application specification file, distributed electronically, and loaded onto the Alinity m System.

Recent studies have shown that heat treatment of 60° C. for 30 minutes only partially inactivates SARS-CoV-2 leaving a low, but still viable concentration of viral particles in solution. To overcome this, the use of 1.4 M Guanidine Isothiocyanate (GITC), and Tris buffer (Abbott Multicollect Tube), with heat inactivation for 60 minutes at 65° C. has been adopted.

It was also shown that GITC alone, even in high concentrations has been shown to only inactivate 50-75% of SARS-CoV-2. It was believed that adding heat inactivation with dilution in Abbott Multicollect tubes would effectively inactivate all viable SARS-CoV-2 viral particles, however, this belief was unable to be verified with cell culture. In addition, aliquoting 300 μl to Abbott Multicollect tubes and heat inactivating, introduces error (due to dilution) and is time consuming as testing volumes and demand for testing has increased.

Materials

GITC/Triton X-100 Media (hereafter in this example, “the disclosed media”):
4M Guanidinium Thiocyanate
0.08M Tris HCl pH 8.0
0.025M EDTA pH 8.0
10% Triton X-100

### Experimental Design

Shielded RNA Standard Curve and Limit of Detection (LOD)

Serial dilutions were created using SeraCare AccuPlex SARS-CoV-2 shielded RNA positive controls. The initial concentration of viral RNA was 5000 copies/mL. This was diluted in GITC/Triton X-100, to provide viral concentrations of 250 copies/mL, 100 copies/mL, 50 copies/mL, and 10 copies/mL. Each dilution was tested on the Abbott Alinity m five times and CN numbers were recorded. A standard curve was generated using average CN values versus viral copy concentration.

Precision

A dilution was created throughout the study using SeraCare AccuPlex SARS-CoV-2 shielded RNA positive control material. To provide low concentration samples for precision study, the initial concentration of 5000 copies/ml was diluted to 100 copies/ml in the tube to be tested. That is 5000 copies/ml was diluted to OTE (1:5 dilution) and again in OTE (1:10 dilution), yielding 100 copies/ml. Ten positive samples were created and subsequently tested on the Abbott Alinity m platform.

Contrived negative samples were also created using SeraCare AccuPlex SARS-CoV-2 shielded RNA negative controls (Human RNase P). The negative control was diluted in the disclosed media and tested.

These samples were stored at 2-8° C. and tested at 24-hour intervals for 72 hours (initial test, with two additional replicate studies). Samples were arranged on the sample rack alternating positive and negative samples to test for carryover.

Patient Samples

Samples being tested using the Abbott m2000 platform were held and following sample preparation on the m2000 were tested using the Abbott Alinity m. Test results were compared from each system and recorded.

Results

Shielded RNA Standard Curve and Limit of Detection (LOD)

All serial dilution samples were detected. Although all concentrations were reliably detected down to 10 copies/ml, to be conservative the empiric limit of detection for this assay utilizing the disclosed media appeared to be 100 copies/ml.

Precision Study

In the precision study utilizing the disclosed media, 20 initial samples were tested (10 positive at 100 copies/ml SARS-CoV-2 RNA, and 10 negative samples) and 20 replicate samples of the same type were tested at 24 hour intervals up to 72 hours. Precision was found to be 98%, with one known positive sample resulting as negative.

Sensitivity: Sensitivity of the Abbott Alinity m SARS-CoV-2 Assay was calculated to be 96%.

Specificity: Specificity of the Abbott Alinity m SARS-CoV-2 Assay was calculated to be 100%.

Positive Predictive Value: Positive Predictive Value of the Alinity m SARS-CoV-2 Assay was calculated to be 100%.

Negative Predictive Value: Negative Predictive Value of the Alinity m SARS-CoV-2 Assay was calculated to be 98%.

Reportable Range:
SARS-CoV-2 Not Detected
SARS-CoV-2 Detected

### Summary

Of the 157 total specimens tested, there was 99% agreement with expected results. Two of the 157 results were discordant (m2000 Positive, Alinity m Negative), and 1 was discordant (m2000 Negative, Alinity m Positive). The two specimens in which the m2000 was positive and the Alinity was negative did not have additional sample to test as a tie breaker, however, the CN value on the m2000 was near or at the limit of detection for both assays and discordant results at this viral load should be expected.

The specimen in which the Alinity m was positive while the m2000 remained negative, was subsequently tested using the Cepheid Infinity SARS-CoV-2 assay and was found to be positive, albeit with a viral load near or at the Limit of Detection for all assays.

No carryover was observed in the precision study.

Specimens were shown to be adequate and acceptable for testing for up to 72 hours if stored at 2-8° C., and had a limit of detection of 10 copies/ml.

### Conclusion

Abbott Alinity m SARS-CoV-2 assay is acceptable for testing patients for SARS-CoV-2 when used with the disclosed media.

### Example 3—Validation and Demonstration of Disclosed Media with Biofire Platform

This example describes the validation and demonstration of acceptable performance of the disclosed media for use with the Biofire® FilmArray® Respiratory Panel 2.1 (RP2.1). The Biofire® Torch System and Biofire® FilmArray® Respiratory Panel 2.1 (RP2.1) are used for the simultaneous qualitative detection and identification of multiple respiratory bacterial and viral nucleic acids.

Materials

GITC/Triton X-100 Media (hereafter in this example, “the disclosed media”):
4M Guanidinium Thiocyanate
0.08M Tris HCl pH 8.0
0.025M EDTA pH 8.0
10% Triton X-100

Method

The FilmArray® Respiratory Panel 2.1 (RP2.1) is a multiplexed nucleic acid test intended for use with FilmArray® 2.0 or FilmArray® Torch Systems for the simultaneous qualitative detection and identification of multiple respiratory viral and bacterial nucleic acids in nasopharyngeal swabs (NPS) obtained from individuals suspected of respiratory tract infections. The following organism types and subtypes are identified using the FilmArray® RP2.1:
SARS-CoV-2
Adenovirus
Coronavirus 229E
Coronavirus HKU1
Coronavirus NL63
Coronavirus OC43
Human Metapneumovirus
Human Rhinovirus/Enterovirus
Influenza A, including subtypes H1, H1-2009, and H3
Influenza B
Parainfluenza 1
Parainfluenza 2
Parainfluenza 3
Parainfluenza 4
Respiratory Syncytial Virus
Chlamydia pneumoniae
Mycoplasma pneumoniae
Bordetella pertussis (ptxP)
Bordetella parapertussis (IS1001)

The FilmArray® RP2.1 is a closed system disposable that stores all the necessary reagents for sample preparation, reverse transcription, polymerase chain reaction (PCR), and detection in order to isolate, amplify, and detect nucleic acids from multiple respiratory pathogens within a single NPS specimen. After sample collection, the user injects hydration solution, and sample combined with sample buffer into the pouch, places the pouch into a FilmArray® instrument module, and starts a run. The entire run process takes about 50 minutes. Additional details can be found in the FilmArray® operator's manual.

During a run, the FilmArray® system:
Lyses the sample by agitation (bead beating)
Extracts and purifies all nucleic acids from the sample using magnetic bead technology a Performs nested multiplex PCR by first, performing reverse transcription and a single, large volume, multiplexed reaction (PCR1). Then, it performs multiple singleplex second-stage PCR reactions (PCR2) to amplify sequences within the PCR1 products.
Uses endpoint melting curve data to detect and generate a result for each target assay on the FilmArray® RP2.1 panel

The validation followed the VTM protocol suggested by the manufacturer. Zeptometrix QC sample material was pooled and added to an equal volume of Universal Transport Media™ (UTM®) from Copan Diagnostics, and Remel or the disclosed media. To satisfy the requirement of verification of all instrument modules, 64 total tests were run on 2 consecutive days (32 tests per day) using media 2, providing 16 positive results and 48 negative results per assay.

Pooling utilizing UTM was performed on Bays 5-8 for 2 consecutive days. Pooling utilizing media 2 was performed on Bays 1-8 for 2 consecutive days.

Results

All 4 pools, tested in each bay all performed as expected with 100% concordance with expected results. Replicate studies the following day also performed with 100% concordance with expected results.

Sensitivity: Sensitivity of the Biofire FilmArray RP2.1 Assay was calculated to be 100%.

Specificity: Specificity of the Biofire FilmArray RP2.1 Assay was calculated to be 100%.

Positive Predictive Value: Positive Predictive Value of the Biofire FilmArray RP2.1 Assay was calculated to be 100%.

Negative Predictive Value: Negative Predictive Value of the Biofire FilmArray RP2.1 Assay was calculated to be 100%.

Reportable Range:
SARS-CoV-2—Detected/Not Detected
Adenovirus—Detected/Not Detected
Coronavirus 229E—Detected/Not Detected
a Coronavirus HKU1—Detected/Not Detected
Coronavirus NL63—Detected/Not Detected
Coronavirus OC43—Detected/Not Detected
Human Metapneumovirus—Detected/Not Detected
Human Rhinovirus/Enterovirus—Detected/Not Detected
Influenza A, including subtypes H1, H1-2009, and H3—Detected/Not Detected
Influenza B—Detected/Not Detected
Parainfluenza 1—Detected/Not Detected
Parainfluenza 2—Detected/Not Detected
Parainfluenza 3—Detected/Not Detected
Parainfluenza 4—Detected/Not Detected
Respiratory Syncytial Virus—Detected/Not Detected
Chlamydia pneumoniae—Detected/Not Detected
Mycoplasma pneumoniae—Detected/Not Detected
Bordetella pertussis (ptxP)—Detected/Not Detected
Bordetella parapertussis (IS1001)—Detected/Not Detected

### Summary

All results were acceptable, showing no day-to-day variation between results. Using the proposed organism pooling scheme provided by the manufacturer, each Sample Pool contained 5-6 different organisms that were “Detected” by RP2.1. All other targets in each Sample Pool were “Not Detected”. In total, when testing the UTM, there were 8 positive (“Detected”) results and 24 negative (“Not Detected”) results for each organism, yielding a total of 736/736 acceptable results (32 tests×23 organisms tested). During testing of the media 2, there were 16 positive (“Detected”) results and 48 negative (“Not Detected”) results for each organism, yielding a total of 1,472/1,472 acceptable results (64 tests×23 organisms tested).

### Conclusion

The FilmArray® Respiratory Panel 2.1 (RP2.1) used with the Biofire® FilmArray® Torch system was an acceptable method of testing for the simultaneous qualitative detection and identification of multiple respiratory viral and bacterial nucleic acids from individuals suspected of respiratory tract infections when nasopharyngeal swabs were collected using standard technique and immediately placed in 1-3 mL of a disclosed media for testing.

### Example 4—Validation and Demonstration of Disclosed Media with Abbot m2000 SARS-CoV-2 Assay

This example describes the validation and demonstration of acceptable performance of the disclosed media for use in the Abbot m2000 SARS-CoV-2 Assay. Abbott m2000 detects target sequences using target-specific fluorescent-labeled oligonucleotide probes. It consists of a sample preparation unit, the Abbott m2000sp, and an amplification and detection unit, the Abbott m2000rt. Application parameters specific to each assay are contained on an assay-specific application specification file, distributed electronically, stored on portable media and loaded onto the Abbott m2000sp and Abbott m2000rt instruments.

Materials

GITC/Triton X-100 Media (hereafter in this example, “the disclosed media”):
4M Guanidinium Thiocyanate
0.08M Tris HCl pH 8.0
0.025M EDTA pH 8.0
10% Triton X-100

### Experimental Design

Assay Method

The Abbot RealTime (rt) SARS-CoV-2 assay is a real-time polymerase chain reaction (PCR) test intended for the qualitative detection of nucleic acid from the SARS-CoV-2 in nasopharyngeal and oropharyngeal swabs from individuals with signs and symptom of infection who are suspected of COVID-19. Results are for the identification of SARS-CoV-2 RNA. The SARS-CoV-2 RNA is generally detectable in nasopharyngeal and oropharyngeal swabs during the acute phase of infection. Positive results are indicative of active infection. Negative results do not preclude SARS-CoV-2 infection and should not be used as the sole basis for patient management decisions. Negative results must be combined with clinical observations, patient history and epidemiological information. The Abbott RealTime SARS-CoV-2 assay can be assessed on the Abbott m2000 System. The SARS-CoV-2 primer and probe sets are designed to detect RNA from SARS-CoV-2 in nasopharyngeal and oropharyngeal swabs from patients with signs and symptoms of infection who are suspected of COVID-19.

An RNA sequence that is unrelated to the SARS-CoV-2 target sequence is introduced into each specimen at the beginning of sample preparation. This unrelated RNA sequence (pumpkin genes) is simultaneously amplified by RT-PCR and serves as an internal control to demonstrate that the process has proceeded correctly for each sample.

The Abbott RealTime SARS-CoV-2 assay detects the SARS-CoV-2 virus and internal control target sequences through the use of target-specific fluorescent-labeled oligonucleotide probes. The probes do not generate a signal unless they are specifically bound to the amplified product. The two SARS-CoV-2-specific probes are labeled with the same fluorophore and the internal control-specific probe is labeled with a different fluorophore, thus allowing for simultaneous detection of both SARS-CoV-2 and internal control amplified products in the same reaction well.

The Abbott m2000 System includes a sample preparation unit, the Abbott m2000sp, and an amplification and detection unit, the Abbott m2000rt. Application parameters specific to the Abbott RealTime SARS-CoV-2 assay are contained on an assay-specific application file, distributed electronically, stored on portable media and loaded onto the Abbott m2000sp and Abbott m2000rt instruments.

Shielded RNA Standard Curve and Limit of Detection (LOD)

Serial dilutions were created using SeraCare AccuPlex SARS-CoV-2 shielded RNA positive controls. The initial concentration of viral RNA was 5000 copies/mL. This was diluted in the disclosed media, to provide viral concentrations of 250 copies/mL, 100 copies/mL, 25 copies/mL, and 10 copies/mL. Each dilution was tested on the Abbott m2000 five times and CN numbers were recorded. A standard curve was generated using average CN values versus viral copy concentration.

Correlation Study

NP samples that were collected were initially tested using Abbott IDNow SARS-CoV-2 Assay, and Cepheid GeneXpert Infinity SARS-CoV-2 Assay, and subsequently tested for correlation on the Abbott m2000 SARS-CoV-2 assay utilizing the disclosed media.

The patients' initial samples were collected upon admission in Universal Transport Media (UTM) and tested on either the Abbott IDNOW or the Cepheid GeneXpert Infinity. Later, both NP and Nasal swabs were again collected and immediately placed in the disclosed media and transported to the microbiology laboratory. The samples were then loaded on the m2000. CN values and results were recorded.

Spiked Samples and Precision

Serial dilutions were created using SeraCare AccuPlex SARS-CoV-2 shielded RNA positive controls.

To provide middle and low concentration samples, the initial concentration of 5000 copies/ml was diluted to 500 copies/ml, 300 copies/ml, 100 copies/ml, and 50 copies/ml. Five spiked samples (20 samples total) were created at each concentration and tested on the Abbott m2000 instrument. These samples were stored at 2-8° C. and tested at 24-hour intervals for 72 hours (initial test, with two additional replicate studies).

Contrived negative samples were also created using SeraCare AccuPlex SARS-CoV-2 shielded RNA negative controls. The negative control was diluted in the disclosed media and tested. The negative samples were also stored at 2-8° C. and tested at 24-hour intervals for 72 hours (initial test, with two additional replicate studies).

Results

Shielded RNA Standard Curve and Limit of Detection (LOD)

All serial dilution samples were detected, except for two of five samples at 10 copies/ml. 10 copies/ml is the empiric limit of detection for this assay utilizing the disclosed media.

Correlation Study

There were 26 samples of known SARS-CoV-2 positive patients tested. A combination of bilateral nasal and NP collection were studied. Of those, one bilateral nasal collection was found to be negative, and the other 25 samples were found to be positive (CN range 11.01 to 29.21) in agreement with other SARS-CoV-2 testing methodologies.

Nasal collection contained notably less viral RNA than the corresponding NP collection. CN values for bilateral nasal collections were much higher than those of the NP swab on the same patient. The only nasal collection that was negative could have been due to low viral count with this collection method, as the corresponding NP collection seemed to have a high CN value (26.97) and caused the nasal collection to fall below the limit of detection for this assay.

Precision Study

In the precision study utilizing the disclosed media, 90 replicate tests were performed with only one (1%) out of agreement with the expected results. 89 of 90 replicate tests (99%) agreed with the original expected result. The single outlier sample was tested 10 times, all of which retested positive, showing that the single test out of agreement may have been due to instrument failure, or experimental failure.

During the precision study, samples were stored at 2-8° C. for 72 hours and showed similar CN values with each test, showing that samples are stable for at least 72 hours after collection at 2-8° C.

Sensitivity: Sensitivity of the Abbott m2000 SARS-CoV-2 Assay was calculated to be 98%.

Specificity: Specificity of the Abbott m2000 SARS-CoV-2 Assay was calculated to be 100%.

Positive Predictive Value: Positive Predictive Value of the Abbott m2000 SARS-CoV-2 Assay was calculated to be 100%.

Negative Predictive Value: Negative Predictive Value of the Abbott m2000 SARS-CoV-2 Assay was calculated to be 96%.

Reportable Range:
SARS-CoV-2 Negative
SARS-CoV-2 Positive

### Summary

Of the 158 total specimens tested, only 2 samples did not agree with the expected results. The one sample in disagreement may have been due to experimental or instrument error, while the other showed nasal collection to be inferior to nasopharyngeal collection.

Specimens were shown to be adequate and acceptable for testing for up to 72 hours if stored at 2-8° C., and has a limit of detection of 10 copies/ml. In all studies, no carryover was observed.

### Conclusion

Abbott m2000 SARS-CoV-2 assay while utilizing the disclosed media is acceptable for testing patients for SARS-CoV-2.

### Example 5—Validation and Demonstration of Disclosed Media with Abbot m2000 SARS-CoV-2 Assay for Terminal Inactivation

This example describes the validation and demonstration of acceptable performance of the disclosed media for use in the Abbot m2000 SARS-CoV-2 Assay as the terminal inactivating step when samples are initially collected in Universal Transport Media™ (UTM®) from Copan Diagnostics, and Remel. Abbott m2000 detects target sequences using target-specific fluorescent-labeled oligonucleotide probes. It consists of a sample preparation unit, the Abbott m2000sp, and an amplification and detection unit, the Abbott m2000rt. Application parameters specific to each assay are contained on an assay-specific application specification file, distributed electronically, stored on portable media and loaded onto the Abbott m2000sp and Abbott m2000rt instruments.

Materials

GITC/Triton X-100 Media (hereafter in this example, “the disclosed media”):
4M Guanidinium Thiocyanate
0.08M Tris HCl pH 8.0
0.025M EDTA pH 8.0
10% Triton X-100

### Experimental Design

Assay Method

The Abbot RealTime (rt) SARS-CoV-2 assay is a real-time polymerase chain reaction (PCR) test intended for the qualitative detection of nucleic acid from the SARS-CoV-2 in nasopharyngeal and oropharyngeal swabs from individuals with signs and symptom of infection who are suspected of COVID-19. Results are for the identification of SARS-CoV-2 RNA. The SARS-CoV-2 RNA is generally detectable in nasopharyngeal and oropharyngeal swabs during the acute phase of infection. Positive results are indicative of active infection. Negative results do not preclude SARS-CoV-2 infection and should not be used as the sole basis for patient management decisions. Negative results must be combined with clinical observations, patient history and epidemiological information. The Abbott RealTime SARS-CoV-2 assay can be assessed on the Abbott m2000 System. The SARS-CoV-2 primer and probe sets are designed to detect RNA from SARS-CoV-2 in nasopharyngeal and oropharyngeal swabs from patients with signs and symptoms of infection who are suspected of COVID-19.

An RNA sequence that is unrelated to the SARS-CoV-2 target sequence is introduced into each specimen at the beginning of sample preparation. This unrelated RNA sequence (pumpkin genes) is simultaneously amplified by RT-PCR and serves as an internal control to demonstrate that the process has proceeded correctly for each sample.

The Abbott RealTime SARS-CoV-2 assay detects the SARS-CoV-2 virus and internal control target sequences through the use of target-specific fluorescent-labeled oligonucleotide probes. The probes do not generate a signal unless they are specifically bound to the amplified product. The two SARS-CoV-2-specific probes are labeled with the same fluorophore and the internal control-specific probe is labeled with a different fluorophore, thus allowing for simultaneous detection of both SARS-CoV-2 and internal control amplified products in the same reaction well.

The Abbott m2000 System includes a sample preparation unit, the Abbott m2000sp, and an amplification and detection unit, the Abbott m2000rt. Application parameters specific to the Abbott RealTime SARS-CoV-2 assay are contained on an assay-specific application file, distributed electronically, stored on portable media and loaded onto the Abbott m2000sp and Abbott m2000rt instruments.

Shielded RNA Standard Curve and Limit of Detection (LOD)

Serial dilutions were created using SeraCare AccuPlex SARS-CoV-2 shielded RNA positive controls. The initial concentration of viral RNA was 5000 copies/mL. This was diluted in the disclosed media, to provide viral concentrations of 250 copies/mL, 100 copies/mL, 25 copies/mL, and 10 copies/mL. Each dilution was tested on the Abbott m2000 five times and CN numbers were recorded. A standard curve was generated using average CN values versus viral copy concentration. LOD was used from the previous study, as limit of detection will not vary.

Spiked Samples and Precision

A dilution was created throughout the study using SeraCare AccuPlex SARS-CoV-2 shielded RNA positive control material. To provide low concentration samples, the initial concentration of 5000 copies/ml was diluted to 125 copies/ml in the tube to be tested. That is 5000 copies/ml was diluted to UTM (1:5 dilution) and again in the disclosed media (3:23 dilution), yielding 125 copies/ml. Ten samples were created and subsequently tested on the Abbott m2000 platform. These samples were stored at 2-8° C. and tested at 24-hour intervals for 72 hours (initial test, with two additional replicate studies).

Contrived negative samples were also created using SeraCare AccuPlex SARS-CoV-2 shielded RNA negative controls (Human RNase P). The negative control was diluted in the disclosed media and tested. The negative samples were also stored at 2-8° C. and tested at 24-hour intervals for 72 hours (initial test, with two additional replicate studies).

Results

Shielded RNA Standard Curve and Limit of Detection (LOD)

All serial dilution samples were detected, except for two of five samples at 10 copies/ml. 10 copies/ml is the empiric limit of detection for this assay utilizing the disclosed media. This LOD was used for the current study of inactivating SARS-CoV-2 initially collected in UTM, in the disclosed media.

Precision Study

In the precision study utilizing the disclosed media as the terminal inactivating step from specimens collected in UTM, 20 initial samples were tested (10 positive at 125 copies/ml SARS-CoV-2 RNA, and 10 negative samples) and 40 (same 10 positive and 10 negatives tested) replicate tests were performed 100% agreement with expected results.

During the precision study, samples were stored at 2-8° C. for 72 hours and showed similar CN values with each test, showing that samples are stable for at least 72 hours after collection at 2-8° C.

Sensitivity: Sensitivity of the Abbott m2000 SARS-CoV-2 Assay was calculated to be 100%.

Specificity: Specificity of the Abbott m2000 SARS-CoV-2 Assay was calculated to be 100%.

Positive Predictive Value: Positive Predictive Value of the Abbott m2000 SARS-CoV-2 Assay was calculated to be 100%.

Negative Predictive Value: Negative Predictive Value of the Abbott m2000 SARS-CoV-2 Assay was calculated to be 100%.

Reportable Range:
SARS-CoV-2 Negative
SARS-CoV-2 Positive

### Summary

Of the 60 total specimens tested, there was 100% agreement with expected results.

Specimens were shown to be adequate and acceptable for testing for up to 72 hours if stored at 2-8° C., and had a limit of detection of 10 copies/ml. In all studies, no carryover was observed.

### Conclusion

Abbott m2000 SARS-CoV-2 assay while utilizing the disclosed media as the terminal inactivating step from specimens initially collected in UTM is acceptable for testing patients for SARS-CoV-2.

### Example 6—Validation and Demonstration of Disclosed Media with Cepheid Platform

This example describes the validation and demonstration of acceptable performance of the disclosed media for use in the Cepheid Infinity SARS-CoV-2 assay, for a more streamline workflow, to limit errors due to multiple media types being used, and to ensure safety in the laboratory. The Cepheid Xpert® SARS-CoV-2 Assay, performed on the Cepheid GeneXpert® Infinity System, is an automated real-time PCR assay for the in vitro qualitative detection of nucleic acid from SARS-CoV-2 in either nasopharyngeal swab and/or nasal wash/aspirate specimens collected from individuals suspected of COVID-19 by their healthcare provider. Testing is limited to laboratories certified under the Clinical Laboratory Improvement Amendments of 1988 (CLIA), 42 U.S.C. § 263a, to perform high and moderate complexity tests. The term “qualified laboratories” refers to laboratories in which all users, analysts, and any person reporting results from use of this device are proficient in performing real-time RT-PCR assays.

The GeneXpert Infinity System automates and integrates sample purification, nucleic acid amplification, and detection of the target sequence in simple or complex samples using real-time PCR assays. The system consists of a fully automated instrument, person and preloaded software for running tests and viewing the results. The system requires the use of single-use disposable cartridges that hold the RT-PCR reagents and host the RT-PCR process. Because the cartridges are self-contained, cross-contamination between samples is minimized with careful attention to procedure. The Xpert Xpress SARS-CoV-2 test includes reagents for the detection of RNA from SARS-CoV-2 in nasopharyngeal swab specimens. A Sample Processing Control (SPC) and a Probe Check Control (PCC) are also included in the cartridge utilized by the GeneXpert instrument. The SPC is present to control for adequate processing of the sample and to monitor for the presence of potential inhibitor(s) in the RT-PCR reaction. The SPC also ensures that the RT-PCR reaction conditions (temperature and time) are appropriate for the amplification reaction and that the RT-PCR reagents are functional. The PCC verifies reagent rehydration, PCR tube filling, and confirms that all reaction components are present in the cartridge including monitoring for probe integrity and dye stability.

Materials

GITC/Triton X-100 Media (hereafter in this example, “the disclosed media”):
4M Guanidinium Thiocyanate
0.08M Tris HCl pH 8.0
0.025M EDTA pH 8.0
10% Triton X-100

### Experimental Design

Shielded RNA Standard Curve and Limit of Detection (LOD)

Serial dilutions were created using SeraCare AccuPlex SARS-CoV-2 shielded RNA positive controls. The initial concentration of viral RNA was 5000 copies/mL. This was diluted in the disclosed media, to provide viral concentrations of 100 copies/mL, 50 copies/mL, 25 copies/mL, and 10 copies/mL. Each dilution was tested on the Cepheid Infinity SARS-CoV-2 assay five times and CT numbers were recorded. A standard curve was generated using average CT values versus viral copy concentration.

Spiked Samples and Precision

, Serial dilutions were created using SeraCare AccuPlex SARS-CoV-2 shielded RNA positive controls.

To provide middle and low concentration samples, the initial concentration of 5000 copies/ml was diluted to 500 copies/ml, and 100 copies/ml. Five spiked samples (10 samples total) were created at each concentration and tested on the Cepheid Infinity SARS-CoV-2 assay instrument. These samples were stored at 2-8° C. and tested at 24-hour intervals for 72 hours (initial test, with two additional replicate studies).

Contrived negative samples were also created using SeraCare AccuPlex SARS-CoV-2 shielded RNA negative controls. The negative control was diluted in the disclosed media and tested. The negative samples were also stored at 2-8° C. and tested at 24-hour intervals for 72 hours (initial test, with two additional replicate studies).

Results

Shielded RNA Standard Curve and Limit of Detection (LOD)

Dilutions that were created at 10 and 25 copies/ml were detected, albeit unreliably. At 50 copies/ml all samples were detected, except for one N2 gene on sample 4 yielding a presumptive positive result. The empiric LOD was calculated to be 50 copies/ml.

Precision Study

In the precision study utilizing the disclosed media, 60 tests were performed. 20 (10 positive and 10 negative) replicate tests were performed at 24 hours and again at 48 hours. All initial tests and replicates agreed with the expected results, yielding a precision value of 100% agreement.

During the precision study, samples were stored at 2-8° C. for 72 hours and showed similar CT values with each test, showing that samples are stable for at least 72 hours after collection at 2-8° C.

Sensitivity: Sensitivity of the Abbott m2000 SARS-CoV-2 Assay was calculated to be 100%.

Specificity: Specificity of the Abbott m2000 SARS-CoV-2 Assay was calculated to be 100%.

Positive Predictive Value: Positive Predictive Value of the Abbott m2000 SARS-CoV-2 Assay was calculated to be 100%.

Negative Predictive Value: Negative Predictive Value of the Abbott m2000 SARS-CoV-2 Assay was calculated to be 100%.

Reportable Range:
SARS-CoV-2 Negative
SARS-CoV-2 Positive

### Summary

Of the 60 samples that were tested, all samples agreed with expected results.

Specimens were shown to be adequate and acceptable for testing for up to 72 hours if stored at 2-8° C., and had a limit of detection of 50 copies/ml. In all studies, no carryover was observed.

### Conclusion

Cepheid Infinity SARS-CoV-2 assay while utilizing the disclosed media is acceptable for testing patients for SARS-CoV-2.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

All patents, patent applications, provisional applications, and publications referred to or cited herein are incorporated by reference in their entirety, including all figures and tables, to the extent they are not inconsistent with the explicit teachings of this specification.

What is claimed is:

1. A method of storing a biological sample, comprising
obtaining from a subject a biological sample that contains or is believed to contain a pathogenic organism, and contacting the sample with a medium, consisting of:
(a) a chaotropic agent in an amount of about 3 M to about 5 M;
(b) a buffer in an amount of about 0.4 M to about 0.5 M at about pH 8.0;
(c) a chelating agent in an amount of about 15 mM to about 35 mM;
(d) a detergent in an amount of about 5% to about 15%;
(e) water; and
(f) optionally, N-acetyl cysteine.

2. The method of claim 1, wherein the biological sample is stored in the medium for at least 24 hours.

3. The method of claim 1, wherein the medium stabilizes DNA or RNA in the biological sample for at least 72 hours.

4. The method of claim 1, wherein any pathogenic organism in the biological sample is inactivated by the medium.

5. The method of claim 1, wherein the biological sample is selected from sputum, saliva mucus, blood, plasma, serum, tissue, and a combination thereof.

6. The method of claim 1, wherein the sample is obtained by a nasal swab, a nasopharyngeal swab, an oropharyngeal swab, or a bronchoalveolar lavage (BAL).

7. The method of claim 1, wherein the pathogenic organism is a virus, a bacterium, or a parasite.

8. The method of claim 1, wherein the biological sample is contacted with the medium upon obtaining the biological sample and not placed, transported, or stored in a different medium beforehand.

9. The method of claim 1 further comprising detecting or quantifying nucleic acids in the biological sample after it has been contacted with the microbiology transport medium.

10. The method of claim 1, wherein the chaotropic agent is selected from the group consisting of guanidine isothiocyanate, urea, lithium perchlorate, lithium acetate, phenol, thiourea, and guanidium chloride.

11. The method of claim 1, wherein the chaotropic agent is present in an amount of about 4 M.

12. The method of claim 1, wherein the buffer is selected from the group consisting of Tris, sodium citrate/citrate buffer, L-glycine, acetate, borate, diethanolamine, carbonate (sodium), phosphate, MOPS (2-(N-morpholino)ethanesulfonic acid), bis-tris methane, ADA (N-(2-acetamido)iminodiacetic acid), bis-tris propane, PIPES (piperazine-N,N′-bis(2-ethanesulfonic acid)), ACES (N-(2-acetamido)-2-aminoethanesulfonic acid), MOPSO (3-morpholinopropanesulfonic acid), cholamine chloride, BES (N,N-Bis(2-hydroxyethyl)-2-aminoethanesulfonic acid), TES (2-[(2-hydroxy-1,1-bis(hydroxymethyl)ethyl)amino]ethanesulfonic acid), HEPES (4-(2-hydroxyethyl)-1-piperazineethanesulfonic acid), DIPSO (3-(N,N-Bis[2-hydroxyethyl]amino)-2-hydroxypropanesulfonic acid), MOBS (4-(N-morpholino)butanesulfonic acid), acetamindoglycine, TAPSO (2-hydroxy-3-[tris(hydroxymethyl)methylamino]-1-propanesulfonic acid), TEA (N,N-diethylethanamine), POPSO (piperazine-1,4-bis(2-hydroxypropanesulfonic acid) dihydrate), HEPPSO (4-(2-Hydroxyethyl)piperazine-1-ethanesulfonic acid), HEPPS (3-[4-(2-Hydroxyethyl)piperazin-1-yl]propane-1-sulfonic acid), tricine, glycinamide, glycylglycine, HEPBS (N-(2-Hydroxyethyl)piperazine-N′-(4-butanesulfonic acid)), bicine, TAPS ([tris(hydroxymethyl)methylamino]propanesulfonic acid), AMPSO (N-(1,1-Dimethyl-2-hydroxyethyl)-3-amino-2-hydroxypropanesulfonic acid), CAPS (N-cyclohexyl-3-aminopropanesulfonic acid), CABS (4-(cyclohexylamino)-1-butanesulfonic acid), and CHES (N-(cyclohexylamino)ethanesulfonic acid).

13. The method of claim 1, wherein the buffer is present in an amount of about 0.4 M.

14. The method of claim 1, wherein the chelating agent is selected from the group consisting of ethylenediaminetetraacetic acid (EDTA); ethyleneglycol-bis(R-aminoethyl)-N,N,N′,N′-tetraacetic acid; ethylene glycol-bis(R-aminoethyl ether)-N,N,N′,N′-tetraacetic acid tetrasodium salt; 1,2-bis(o-aminophenoxy)ethane-N,N,N′,N′-tetraacetic acid; 1,2-bis(2-aminophenoxy)ethane-N,N,N′,N′-tetraacetic acid; and deferoxamine mesylate.

15. The method of claim 1, wherein the chelating agent is present in an amount of about 25 mM.

16. The method of claim 1, wherein the detergent is selected from the group consisting of Triton X-100, lithium dodecyl sulfate, sodium dodecyl sulfate, sodium lauryl sulfate, lithium lauryl sulfate, potassium lauryl sulfate, DDM (n-dodecyl beta-D-maltoside), digitonin, Tween 20, Tween 80, Chaps (3-[(3-Cholamidopropyl)dimethylammonio]-1-propanesulfonate), deoxycholate, cholate, and sarkosyl.

17. The method of claim 1, wherein the detergent is present in an amount of about 10%.

18. The method of claim 1, wherein the water is RNase free and/or DNase water.

19. The method of claim 1, wherein the medium includes N-acetyl cysteine.

20. The method of claim 1, wherein the medium, consists of:
(a) 4 M guanidine isothiocyanate;
(b) 0.4 M Tris hydrochloride (HCl) at about pH 8.0;
(c) 25 mM ethylenediaminetetraacetic acid (EDTA) at about pH 8.0;
(d) 10% Triton X-100;
(e) water; and
(f) optionally, N-acetyl cysteine.
